(19) United States
(12) Patent Application Publication
(10) Pub. No.: US 2014/0275289 A1
(43) Pub. Date: Sep. 18, 2014

(54) CONCENTRATE FOR FORMING WATER-GEL EMULSION MATRIX AND KIT INCLUDING SAME

(71) Applicant: CONCEPT LABORATORIES, INC., Chicago, IL (US)

(72) Inventors: Zoe Weisman, Chicago, IL (US); Jose Torres, Chicago, IL (US)

(73) Assignee: CONCEPT LABORATORIES, INC., Chicago, IL (US)

(21) Appl. No.: 13/841,556

(22) Filed: Mar. 15, 2013

(51) Int. Cl. A61K 8/81; A61Q 19/08; C08K 5/01

(52) U.S. Cl. CPC A61K 8/8147; C08K 5/01; A61Q 19/08
USPC 514/772.6; 524/83; 428/35.7

(57) ABSTRACT

A kit and method for using the kit to make a water-based consumer product containing oil-soluble materials. The kit includes an ampoule containing an anhydrous concentrate including a polymer dispersed with a binding agent containing a surfactant, and zero or more oil soluble ingredients. The polymer preparation preferably is between about 8% and about 50% by weight of the concentrate, and the polymer preparation is configured to be between preferably about 1% and about 5%, by weight, of the consumer product. The method may include the steps of opening the ampoule, depositing the contents of the ampoule into a container, adding a predetermined amount of water to the container, closing the container, and agitating the container for a predetermined amount of time. The ampoule preferably is airtight and opaque, preventing degradation of the active ingredients within the ampoule.

### BACKGROUND

1. Field of the Invention

This invention relates to an emulsion composition and a kit including the composition for use in water-based consumer products, including but not limited to cosmetics.

2. Background of the Invention

Consumers, cosmetologists, and dermatologists recognize the beneficial effects of skin care ingredients like Retinol and Vitamin C. These and other beneficial ingredients are among a class of unstable raw materials that are valued for their efficacy in the laboratory but are criticized in the marketplace due to their short shelf life. Commonly, exposing these ingredients to oxygen, extreme temperatures, or UV light cause the compounds to degrade and lose efficacy. Thus, the beneficial impact of the product is diminished.

Common forms of consumer products, particularly cosmetics, may be creams, emulsions, gels, and serums, each of which is susceptible to the degradation described above. As a result, chemists, manufacturers, and marketers struggle with delivering finished products to the consumer in these common forms that remain effective throughout their shelf-lifes and end-uses.

In addition, one of the greatest challenges companies face is integrating sustainability with economic growth. While “bigger and better” retail cartons look alluring on the retail shelf, packaging components and retail cartons consume energy and resources that make sustainability virtually impossible. The European Union (EU) Sustainable Consumption and Production and Sustainable Industrial Policy Action Plan of 2008 is one example of a governing body encouraging suppliers to prevent or reduce the impact of packaging waste on the environment. With rapid global changes, growing energy demands and natural resource depletion, it is crucial for suppliers to take responsibility for the environmental impact of their products. Historically, the EU leads the way in cosmetic product safety and procedural regulations, followed by California and the rest of the United States. This issue is not limited to cosmetic formulations but also applies, perhaps even more so due to their larger sizes, to other consumer products such as car waxes and cleaners.

Besides the formulation and packaging issues discussed above, manufacturers also are faced with increased production costs, primarily in the form of increased material costs. Manufacturers and distributors also face increased costs for packaging and shipping, primarily due to the increasing weight of “bigger and better” displays and packaging. Conversely, manufacturers and distributors may face pressure from consumers seeking sustainable and reusable forms of packaging to reduce their carbon footprint and to avoid repetitive costs each time a single product is repurchased.

What is needed is a system incorporating a composition that overcomes the drawbacks described above.

### SUMMARY OF THE INVENTION

In one aspect, a kit for making a water-based consumer product containing oil soluble materials includes an ampoule containing an anhydrous concentrate, the concentrate including: a polymer preparation including a polymer dispersed in a carrier oil, the carrier oil containing a surfactant, binding agent; and zero or more oil soluble ingredients, depending on the desired rheology of the finished formulation. The oils act as emollients and contribute to the creamy feel of the final emulsion. The polymer preparation is preferably between about 8% and about 50% by weight of the concentrate, and the polymer preparation is configured to preferably be between about 1% and about 5%, by weight, of the consumer product. The ampoule is preferably airtight and opaque.

The polymer preparation includes a liquid dispersion polymer, a surfactant, a binding agent, and ideally an oil-soluble preservative. These blends typically are sold and marketed as cold-process emulsifying systems to streamline the compounding process for manufacturing large-batch emulsions. These blends reduce the need for heating separate formula phases, homogenizing, sheering, and other time-consuming compounding methods.

Such systems are often comprised of an anhydrous system including ingredients like sodium polyacrylate and trideceth-6. In one embodiment, the polymer preparation further may include isotridecyl isononanoate. In another embodiment, the polymer preparation further may include paraffinium liquidum. Alternatively, the polymer preparation may include sodium polyacrylate, ethylhexyl cocoate, polysorbate 20, and PPG-3 benzyl ether myristate.

The kit also may include a container configured to receive the concentrate and a predetermined quantity of water. The container may include fill-level markings to assist the user in preparing the end product in the proper concentration.

The kit may be used to form a variety of end products, depending on the additional components in the ampoule in addition to the polymer preparation. For example, the kit may be configured to form a cosmetic, and the ampoule may include one or more of an antioxidant, an anti-wrinkle formulation, and a silicone or silicone replacement. In another example, the kit may be configured to form a car wax.

In another aspect, a method of using a kit such as the one described above to make a water-based consumer product containing oil-soluble materials may include the steps of: opening the ampoule, depositing the contents of the ampoule into a container, adding a predetermined amount of water to the container, closing the container, and agitating the container for a predetermined amount of time. Agitation may comprise manual shaking of the container for between about 5 seconds and about 30 seconds, preferably about 10 seconds.

In one embodiment, the concentrate may be configured to make a cosmetic product. In that case, the polymer product may comprise about 10%, by weight, of the concentrate. In another embodiment, the concentrate may be configured to make other consumer products such as a car wax. In that case, the polymer product may comprise about 20%, by weight, of the concentrate.

Aside from allowing consumers to enjoy a more efficacious product and a reduced carbon footprint, they are also given the rare opportunity to be engaged directly with the creation of a product. Considering the disconnect we experience from purchasing products directly off the shelf with minimal knowledge of their origins, this “Shake-Up” process allows the consumer to participate in the construction of their own, personal product. This process is engaging and fun for people of all ages.

These and other features and advantages are evident from the following description of the present invention, with reference to the accompanying drawings.

### BRIEF DESCRIPTION OF THE DRAWINGS

FIG. 1 is a first type of ampoule configured to hold a concentrate.

FIG. 2 is a second type of ampoule configured to hold a concentrate.

FIG. 3 is a container holding a plurality of ampoules.

FIG. 4 is a series of containers configured to receive the contents of an ampoule and water in order to form a final product.

FIG. 5 is a second type of container configured to receive the contents of an ampoule and water in order to form a final product.

FIG. 6 is a depiction of the steps of preparing the final product.

### DETAILED DESCRIPTION OF THE INVENTION

In one embodiment, an anhydrous preparation for use in consumer products, including cosmetics, is described below. The preparation preferably is at least one of freeze, thaw, and temperature stable. As discussed herein, the combination of components, both active and inactive, to which water eventually is to be added may be considered a preparation or a concentrate.

The preparation may include a liquid dispersion polymer, preferably a polyacrylate, as part of an anhydrous solution. In addition, the preparation may include one or more esters. The preparation also may include one or more ethers, which may act as surfactants. The combination of two or more of these components may be referred to herein as a polymer preparation.

As discussed below, the formula may be prepared so that the polyacrylate polymer such as Sodium Polyacrylate, is dispersed in a carrier oil such as mineral oil containing an ionic or quick-inverting surfactant. The Sodium Polyacrylate assists the emulsifier because it also has emulsifying properties. It aids in the inversion of the emulsion by swelling via osmosis and forming a matrix across the oil and water phases, with very little agitation.

The polymer preparation may remain inert until it contacts the added water, at which point the Sodium Polyacrylate interacts with the combination of the binding agent and surfactant. Use of one or more carrier oils may affect the type of final product created, i.e., oils may be used to produce a cream rather than a gel-like feel. Conversely, substantial reduction or omission of the amount of carrier oil used, while retaining the liquid dispersion polymer, the binding agent, and the surfactant, may result in a final product being a gel or having a gel-like consistency.

Upon adding the concentrate to water and further adding mechanical energy to agitate the combination, the surfactant may emulsify the carrier fluid, and the polymer particles may swell by osmosis. A resulting polymer matrix may be created that entraps the emulsified oil, resulting in the formation of a stable emulsion.

Preferably, the ratio of water to concentrate may be between 6:1 and 9:1, depending on the desired viscosity of the final cream, although ratios outside this range are permissible if different viscosities are desired. In the case of formulations for topical application, a ratio of 6:1 may yield a higher viscosity, resulting in a more viscous cream that may have a more occlusive, thick texture. Conversely, a ratio of 9:1 may yield a thinner cream, which may be useful in instances of lightweight lotions, car waxes, etc.

### Polymer Preparation

### Example 1

In one embodiment, the liquid dispersion polymer may be sodium polyacrylate. The preparation further may include isotridecyl isononanoate as an ester and trideceth-6 as the ether or surfactant.

One example of this type of polymer preparation, including relevant proportions of each, may be available commercially from Croda International Plc. or Croda Inc. in the form of a rheology modifier sold under the trademark VISCOPTIMA LV. Exemplary proportions of these components within the polymer preparation, and the functions for which the components are used, are provided in the following table:

| Chemical | Function | Percentage by weight |
|---|---|---|
| Sodium Polyacrylate | Liquid Dispersion | >50% |
| | Polymer | |
| Isotridecyl Isononanoate | Binding agent | 25-50% |
| Trideceth-6 | Surfactant | 1-5% |

### Polymer Preparation

### Example 2

Another suitable polymer preparation may add an emollient to the preparation in order to control the aesthetic feel of the end emulsion. For example, PPG-3 Benzyl Ether Myristate may be used as an emollient. One example of this type of polymer preparation, including relevant proportions of each, may be available commercially from Croda International Plc. or Croda Inc. in the form of a rheology modifier sold under the trademark VISCOPTIMA SE. Exemplary proportions of these components within the polymer preparation, and the functions for which the components are used, are provided in the following table:

| | | |
|---|---|---|
| Chemical | Function | Percentage by weight |

| | | |
|---|---|---|
| Sodium Polyacrylate | Liquid Dispersion | >50% |
| | Polymer | |
| Ethylhexyl Cocoate | Binding agent | 25-50% |
| Polysorbate 20 | Surfactant | 1-5% |
| PPG-3 Benzyl Ether | Emollient | 1-5% |
| Myristate | | |

In addition to adding an emollient, this polymer preparation replaces the trideceth-6 surfactant with polysorbate 20. One benefit over the polymer preparation of Example 1 is that in comparison, the ester substitution in the polymer preparation of Example 2 may exhibit improved electrolyte tolerance and further may permit emulsification of a larger percentage of oils in the final product. For example, while the polymer preparation of Example 1 may be used to emulsify up to 20% oils, the polymer preparation of Example 2 may permit emulsification up to 30% oils.

### Polymer Preparation

### Example 3

A third type of polymer preparation may include aspects of the preparations of Examples 1 and 2, i.e., it may include a surfactant like that of Example 1 in order to retain desirable sheer thinning properties during formation of the final product, while also including an emollient.

The same emollient as in Example 2 may be used in Example 3, or one or more other types of emollients may be used, e.g., paraffinium liquidum, i.e., mineral oil.

One example of this type of polymer preparation, including relevant proportions of each, may be available commercially from Croda International Plc. or Croda Inc. in the form of a rheology modifier sold under the trademark VISCOPTIMA PL. Exemplary proportions of these components within the polymer preparation, and the function for which each component is used, are provided in the following table:

| Chemical | Function | Percentage by weight |
|---|---|---|
| Sodium Polyacrylate | Liquid Dispersion | >50% |
| | Polymer | |
| Paraffinium Liquidum/ | Emollient | 25-50% |
| Mineral Oil | | |
| Trideceth-6 | Surfactant | 1-5% |

It is possible to use other blends comprised of a Sodium Polyacrylate, binding agent, and surfactant to achieve similar results. These blends (and the respective trademarks under which they may be sold) include but are not limited to the following:

### SEPIGEL 305

| | | |
|---|---|---|
| Chemical | Function | Percentage by weight |

| | | |
|---|---|---|
| Polyacrylamide | Liquid Dispersion | >50% |
| | Polymer | |
| C 13-14 Isoparaffin | Emollient | 25-50% |
| Laureth-7 | Surfactant | 1-5% |

### RAPITHIX A-60

| Chemical | Function | Percentage by weight |
|---|---|---|
| Sodium Polyacrylate | Liquid Dispersion | >50% |
| | Polymer | |
| Hydrogenated Polydecene | Emollient | 25-50% |
| Trideceth-6 | Surfactant | 1-5% |

### THORCOVISIC 45

| | | |
|---|---|---|
| Chemical | Function | Percentage by weight |

| | | |
|---|---|---|
| Sodium Polyacrylate | Liquid Dispersion | >50% |
| | Polymer | |
| C 13-14 Isoparaffin | Emollient | 25-50% |
| Laureth-7 | Surfactant | 1-5% |

### INTEGRIGEL O/W-EHS

| | | |
|---|---|---|
| Chemical | Function | Percentage by weight |

| | | |
|---|---|---|
| Sodium Polyacrylate | Liquid Dispersion | >50% |
| | Polymer | |
| Ethylhexyl Stearate | Emollient | 25-50% |
| Trideceth-6 | Surfactant | 1-5% |

Due to the effectiveness of the polymer preparation, it may be possible to create a concentrate in which the polymer preparation is only a minor component. For example, the polymer preparation may range from between about 8% to about 50% of the concentrate. After the addition of water, the polymer preparation may range only from about 1% to about 5% of the final product.

The concentrate may include other components, which may permit it to be customized or tailored to one or more specific uses. In one embodiment, the concentrate may include additional water-soluble ingredients, which may solubilize with the addition of water to the final product.

Preferably, however, the concentrate contains only oil soluble additional components. In that case, the additional components may be dissolved in the oil carrier as part of the concentrate. After the addition of water, agitation then may stimulate the formation of a gel-matrix emulsion, with the additional oil soluble ingredients being suspended within the matrix.

Each additional component of the concentrate preferably is and remains chemically separate from the other components, such that the components may not combine chemically with one another—prior to, during, or after agitation—which may allow each component to retain as much of its independent efficacy as possible.

While polymer preparations of the type described above may be available commercially from one or more sources, it is believed that they heretofore have been used and optimized for quick-forming emulsions only in large-scale compounding projects. In contrast, the application of these preparations as just one component in an end-user system may allow them to experience a highly-effective product, enjoy the process of making their own cream or other final product, and reduce the waste required to deliver effective skin care products to consumers. Whereas commercial systems may use a primary emulsifier, secondary emulsifier and emulsifying waxes ranging from around 15% to 20% or between 4 and 6 grams to create one ounce of product; typical final products here may be formulated using about less than one gram of polymer preparation to yield one ounce.

The types and quantities of additional materials within the concentrate may be determined based on the purpose for which the concentrate may be used. For example, for final preparations intended to be used topically or on the hair, it may be desirable to include oil-soluble beneficial chemicals such as essential fatty acids or tamanu, kukui, lavender, monoi de Tahiti, or argan oils, as well as other components whose uses and relevant concentrations may be known or determined by those of ordinary skill in this art.

For example, in one embodiment, a silky or more luxurious feel may be desired. As such, the anhydrous solution may include one or more of a silicone, an ester, or a silicone replacement. As discussed above with regard to Polymer preparation Example 2, one preferable ester that may be included is PPG-3 Benzyl Ether Myristate, which may be sold by Croda Inc. under the trademark CRODAMOL STS. The concentrate may include this or other silicones, esters, or silicone replacements in order to achieve the desired feeling.

### Concentrate Example 1

### Cosmetic Formulation

As mentioned above, the polymer preparation may be a component in a concentrate whose ultimate use may be as a topical cosmetic product. In a cosmetic product, the polymer preparation may be about 10% by weight of the concentrate. One example of such a cosmetic concentrate is as follows:

| | | | | | Percentage | Percentage |
|---|---|---|---|---|---|---|
| | | | | | of | of Final |
| Ingredient | Purpose | Solubility | Grams | | Concentrate | Formula |
| PPG-3 Benzyl | Rheology | Oil Soluble | 1 | g | 16.90% | 2.0% |
| Ether Myristate | modifier/ | | | | | |
| | Emollient | | | | | |
| Polymer | Emulsifier | Oil Soluble, | 0.75 | g | 12.72% | 1.5% |
| preparation | | containing | | | | |
| (Example 1 | | one water | | | | |
| preparation, | | dispersible | | | | |
| supra) | | agent. | | | | |
| Sunflower Oil | Moisturizer | Oil Soluble | 1.375 | g | 23.33% | 2.75% |
| Preservative(I) | Preservative | Oil Soluble | 0.5 | g | 8.48% | 1.0% |
| Anti-Wrinkle(I) | Anti-Wrinkle | Oil Soluble | 0.5 | g | 8.47% | 1.0% |
| Anti- | Anti-Wrinkle | Oil Soluble | 0.5 | g | 8.48% | 1.0% |
| Wrinkle(II) | | | | | | |
| Anti- | Anti-Wrinkle | Oil Soluble | 0.5 | g | 8.48% | 1.0% |
| Wrinkle(III) | | | | | | |
| Tocopheryl | Antioxidant | Oil Soluble | 0.1 | g | 1.69% | 0.20% |
| Acetate | | | | | | |
| Antioxidant(II) | Antioxidant | Oil Soluble | 0.05 | g | 0.85% | 0.10% |
| Kukui Oil | Moisturizer | Oil Soluble | 0.125 | g | 2.12% | 0.25% |
| Hydroquinone, | Skin- | Oil Soluble | 0.5 | g | 8.48% | 1.0% |
| Tretinoin, | brightening | | | | | |
| Mometasone | | | | | | |
| Furoate | | | | | | |
| | | Totals | 5.9 | g | 100.00% | 11.8% |

For a 50 g total final product, about 44.1 grams of water may be added to this concentrate.

One example of a preservative may be Caprylyl Glycol (and) Phenoxyethanol (and) Hexylene Glycol, which may be sold by Centerchem, Inc. under the trademark DIOCIDE. Other preservatives may be employed, as would be appreciated by one of ordinary skill in the art.

One example of an anti-wrinkle agent may be Propylene Glycol Dicaprate (and) Helianthus Annuus (Sunflower) Seed Cake (and) Hordeum Vulgare (Barley) Extract (and) Cacumis Sativus (Cucumber) Fruit Extract, which may be sold by Barnet Products Corp. under the trademark PHYTOFIX.

Another example of an anti-wrinkle agent may be Caprylic/Capric triglyceride & Lavandula stoechas extract, which may be sold by Barnet Products Corp. under the trademark LAVANDOX.

Still another example of an anti-wrinkle agent may be Caprylic/Capric triglyceride & Hydrogenated vegetable oil & Crithmum maritimum extract, which may be sold by Barnet Products Corp. under the trademark AROPHIRA.

Other anti-wrinkle agents may be employed, as would be appreciated by one of ordinary skill in the art.

Another example of an antioxidant that may be used as part of this concentrate may be the “Super Fruits” Blend sold by VegeTech Corp. As with the anti-wrinkle agents, other antioxidants may be employed, as would be appreciated by one of ordinary skill in the art.

Other cosmetic formulations may be created by varying the additional compounds that are added, the concentrations of those compounds, and the degree to which the concentrate is diluted when forming the final product.

### Concentrate Example 2

### Car Wax Formulation

When combined with other components, the polymer preparation may be employed in various other consumer products, e.g., car waxes. In other consumer product formulations, such as car waxes, the polymer preparation may comprise about 20% by weight of the concentrate. On example of a car wax concentrate may be as follows:

| | | | | | Percentage | Percentage |
|---|---|---|---|---|---|---|
| | | | | | of | of Final |
| Ingredient | Purpose | Solubility | Grams | | Concentrate | Formula |
| Beeswax Milk | Shine/Protectant | Oil Soluble | 7.1 | g | 25.0% | 3.1% |
| Mineral | Solvent | Oil Soluble | 9.4 | g | 33.2% | 4.2% |
| Spirits 66/3 | | | | | | |
| Silicone | High Viscosity | Oil Soluble | 4.1 | g | 14.6% | 1.8% |
| 12,500 cps | Silicone Oil for | | | | | |
| | Shine | | | | | |
| Polymer | Emulsifier | Oil Soluble, | 6.2 | g | 22% | 2.8% |
| preparation | | containing | | | | |
| (Example 1 | | one water | | | | |
| preparation, | | dispersible | | | | |
| supra) | | agent. | | | | |
| Methyl- | Preservative | Miscible in | 0.3 | g | 1.2% | 0.1% |
| chloroiso- | | water | | | | |
| thiazolinone | | | | | | |
| Fragrance | Fragrance | Oil Soluble | 1.1 | g | 4% | 0.5% |
| | | Totals | 28.2 | g | 100% | 12.5% |

For a 226 g (8 oz.) final product, about 197.8 g of water may be added to this concentrate.

One example of the preservative may be available from the DOW CHEMICAL COMPANY under the trademark KATHON.

Concentrate Packaging, Distribution, and Use

Each concentrate including the polymer preparation preferably is an anhydrous formula. The concentrate may be packaged in an opaque, airtight ampoule or tube to ensure stability of the ingredients, such as ampoules 10, 10a shown in FIGS. 1-2. In this way, the concentrate may be insulated from both light and air exposure, substantially reducing degradation of the active ingredients, e.g., the antioxidants, anti-wrinkle agents, etc., in the case of a cosmetic formulation or the shine protector in a car wax compound. In addition, the manufacturer may save in material costs as the packaging required for an ampoule may be significantly smaller than that necessary for a full-sized container of the finished product. Similarly, because the concentrate generally may weigh about 10% of the finished product, the manufacturer may realize significant shipping savings.

In one embodiment, the ampoule 10 may be a component in a kit and may be combined in packaging with a container 12 configured to hold the final product. FIGS. 4-5 illustrate various types and sizes of containers 12, 12a, 12b, 12c that may be used, depending on the type and/or amount of final product to be made. The container may include fill-level markings 14 set at predetermined levels to assist the user in determining how much fluid to add to achieve the desired final product. As seen in FIG. 5, the container may be translucent or include a translucent window 16 proximate the fill-level markings so that the user can determine how much water to add. Alternatively, the container may be opaque, which further may delay degradation of the oil-soluble components. In that case, the fill-level markings may be disposed on the interior of the container.

The kit may include one ampoule or a plurality of ampoules. In the latter case, each ampoule may contain sufficient concentrate to create the recommended amount of end product for use. Alternatively, each ampoule may contain a fraction of the concentrate needed to create the recommended amount; however, these small ampoules may be desirable to create smaller batches of the final product. In that case, the container may include markings to instruct the user in forming a recommended size batch or a differently-sized (e.g., smaller) batch.

In another alternative, as best seen in FIG. 3, the kit may include a container 18 configured to hold a plurality of ampoules, where two ampoules may include different concentrates. For example, one ampoule may hold concentrate for an eye cream, another may hold concentrate for a face cream, another may hold concentrate for a cleanser, etc. In this way, a full skin care system may be provided to the user with even greater reductions in packaging.

Kit also may include a stirring device and an applicator device. Stirring device and applicator may be the same article, or the kit may include separate stirring and application components.

In order to prepare the final product, the user may remove the ampoule from the packaging, open the ampoule, and deposit the concentrate in the container or in another container, as seen in FIG. 6. The user then may add water, which preferably may be any filtered water and more preferably may be bottled water to the concentrate, close the provided container, and shake for a certain length of time, e.g., about 10 seconds. As described above, the addition of mechanical energy to the concentrate may result in the formation of a stable, emulsion that is ready for use.

In one embodiment, the end-consumer is permitted to sanitize and re-use the containers provided in the original kit to reduce waste. The supplier would be able to provide the end-consumer with an additional set of ampoules rather than shipping them an entirely new jar and box, creating a significant waste reduction.

In addition to the cost savings to the manufacturer and distributor (at least some of which may be passed on to the end user), the ampoule may permit the active ingredients to remain inactive and to minimize or avoid the process of degradation until the user is ready to begin using the final product, thereby maximizing the efficacy of that product.

While the foregoing written description of the invention enables one of ordinary skill to make and use what is considered presently to be the best mode thereof, those of ordinary skill will understand and appreciate the existence of variations, combinations, and equivalents of the specific exemplary embodiment and method herein. The invention should therefore not be limited by the above described embodiment and method, but by all embodiments and methods within the scope and spirit of the invention as claimed.

We claim:

1. A kit for making a water-based consumer product containing oil soluble materials, comprising:
an ampoule containing an anhydrous concentrate, the concentrate including:
a preparation containing a polymer such as Sodium Polyacrylate, an inverting surfactant; a binding agent, and
zero or more oil soluble ingredients;
wherein the polymer preparation is between about 8% and about 50% by weight of the concentrate; and
wherein the polymer preparation is configured to be between about 1% and about 5%, by weight, of the consumer product.

2. The kit of claim 1, wherein the ampoule is airtight.

3. The kit of claim 1, wherein the ampoule is opaque or UV coated.

4. The kit of claim 1, wherein the polymer preparation includes:
sodium polyacrylate and trideceth-6.

5. The kit of claim 4, wherein the polymer preparation further includes isotridecyl isononanoate.

6. The kit of claim 4, wherein the polymer preparation further includes paraffinium liquidum.

7. The kit of claim 1, wherein the polymer preparation includes:
sodium polyacrylate, ethylhexyl cocoate, polysorbate 20, and PPG-3 benzyl ether myristate.

8. The kit of claim 1, further including a container configured to receive the concentrate and a predetermined quantity of water.

9. The kit of claim 8, wherein the container includes fill-level markings.

10. The kit of claim 1, further comprising at least one antioxidant.

11. The kit of claim 1, wherein the concentrate is configured to form a cosmetic.

12. The kit of claim 1, wherein the concentrate is configured to form a car wax.

13. A method of using a kit to make a water-based consumer product containing oil soluble materials, the kit comprising:
an ampoule containing an anhydrous concentrate, the concentrate including:
a polymer preparation including a polymer dispersed in a carrier oil, the carrier oil containing an inverting surfactant; and
one or more oil soluble ingredients;
wherein the polymer preparation is between about 8% and about 50% by weight of the concentrate; and
wherein the polymer preparation is configured to be between about 1% and about 5%, by weight, of the consumer product;
the method comprising:
opening the ampoule;
depositing the contents of the ampoule into a container;
adding a predetermined amount of water to the container;
closing the container; and
agitating the container for a predetermined amount of time.

14. The method of claim 13, wherein the predetermined amount of time is between about 5 seconds and about 30 seconds.

15. The method of claim 13, wherein the predetermined amount of time is about 10 seconds.

16. The method of claim 13, wherein the polymer preparation includes:
sodium polyacrylate, isotridecyl isononanoate, and trideceth-6.

17. The method of claim 13, wherein the polymer preparation includes:
sodium polyacrylate, ethylhexyl cocoate, polysorbate 20, and PPG-3 benzyl ether myristate.

18. The method of claim 13, wherein the polymer preparation includes:
sodium polyacrylate, paraffinium liquidum, and trideceth-6.

19. The method of claim 13, wherein the polymer preparation includes any sodium polyacrylate, emollient, and surfactant.

20. The method of claim 13, wherein the concentrate is configured to make a cosmetic product and further wherein the polymer product comprises about 10%, by weight, of the concentrate.

21. The method of claim 13, wherein the concentrate is configured to make a car wax and further wherein the polymer product comprises about 20%, by weight, of the concentrate.
